(19) United States
(12) Patent Application Publication
(10) Pub. No.: US 2026/0013997 A1
(43) Pub. Date: Jan. 15, 2026

(54) METHODS, DEVICES, AND SYSTEMS FOR TREATING BONE DEFECTS

(71) Applicant: The Regents of the University of Michigan, Ann Arbor, MI (US)

(72) Inventors: Alexis Donneys, Ann Arbor, MI (US); Kenneth Kozloff, Ann Arbor, MI (US)

(21) Appl. No.: 19/112,886

(22) PCT Filed: Sep. 19, 2023
(86) PCT No.: PCT/US23/74545

§ 371 Date: Mar. 18, 2025

Related U.S. Application Data
(60) Provisional application No. 63/376,170, filed on Sep. 19, 2022.

(51) Int. Cl. A61F 2/46; A61F 2/28; A61F 2/30

(52) U.S. Cl. CPC A61F 2/4601; A61F 2002/2835; A61F 2002/30065; A61F 2002/30738; A61F 2002/465

(57) ABSTRACT

Provided herein are devices, systems, and methods of treating bone defects by wrapping thermoplastics around bone and molding thermoplastic to bone. Also provided herein are a sonotrode coupler and system used to perform the method. The technology provides devices, systems, and methods for containment of graft materials, matrices, and bone regenerative therapeutics and prevents surrounding tissue encroachment for guided bone regeneration.

The present application claims priority to U.S. Provisional Patent Application Ser. No. 63/376,170, filed Sep. 19, 2022, the disclosure of which is herein incorporated by reference in its entirety.

### FIELD

Provided herein are devices, systems, and methods of treating bone defects by wrapping thermoplastics around bone and molding thermoplastic to bone. Also provided herein are a sonotrode coupler and system used to perform the method. The technology provides devices, systems, and methods for containment of graft materials, matrices, and bone regenerative therapeutics and prevents surrounding tissue encroachment for guided bone regeneration.

### BACKGROUND

Large segmental defects occurring in bone are very challenging surgical reconstructive dilemmas. The emergence of bone regenerative therapeutics, such as bone morphogenic protein, facilitate avenues for the potential healing of these defects, however, the delivery of these therapeutics remains challenging owed to the inability to fully contain the therapeutic at the site of intended action. This problem also occurs with the implantation of bone grafts, matrices, such as mineralized collagen matrix or woven absorbable collagen sponges, or combinations of bone regeneration therapeutics delivered on these implant materials (bone grafts and/or matrices). Methods of guided bone regeneration that dictate the successful geometry of the regenerate bone bridging construct and prevent unwanted ectopic bone formation in other surrounding tissues outside of the intended site, such as nerves, tendons, and muscles, are needed.

### SUMMARY

Provided herein are devices, systems, and methods of treating bone defects by wrapping thermoplastics around bone and molding thermoplastic to bone. Also provided herein are a sonotrode coupler and system used to perform the method. The technology provides devices, systems, and methods for containment of graft materials, matrices, and bone regenerative therapeutics and prevents surrounding tissue encroachment for guided bone regeneration.

For example, in some embodiments a bone defect is contained within a thermoplastic wrap or sleeve and heat (e.g., provided via one or more of ultrasound energy) is used to mold the thermoplastic to the bone, containing the defect, providing mechanical structure to contain the position and shape of the bone region containing the defect, and facilitating healing of the defect, while allowing unobstructed or substantially unobstructed imaging (e.g., X-ray, MRI, CT, bone scan, etc.) of the bone region. In some embodiments, an ultrasonic device configured to deliver ultrasound energy to the contained bone region is utilized to provide efficient thermoforming of the thermoplastic to the bone. However, any heating source may be used, including but not limited to, electrically heated probes, fluid heated probes, warm or hot air, and the like. Additionally, these heating sources may be used singly, or in a combined manner. Ultrasonic/piezo-electric devices have been commercially developed and clinically adopted in the osteosynthesis market. These devices allow surgeons to apply safe and controlled heat and vibratory energy into the surgical field. These devices convert high-frequency ultrasonic energy into acoustic vibrations that can be used to generate localized heat.

Provided herein is a device that combines localized heat with mechanical pressure for thermowelding of a thermoplastic to bone. Thermoplastics come in a variety of sizes allowing for the treatment of bone defects in small and large animals or of varying size within an individual animal. Welding a thermoplastic to bone finds use in research, pre-clinical, and clinical applications. Such use of thermoplastics provides uniformity and standardization for bone graft treatment as well as providing a system for drug delivery in both clinical and research settings. Holding bone defects together with the mechanical properties of thermoplastics prevents unwanted fragment migration. Thermoplastics have several properties that facilitate vascular ingrowth and nutrient exchange. In some embodiments, the systems and methods employ irradiated and stretched “heat-shrink” thermoplastics, that conform back to their unstretched geometry when they are heated. This provides a tightly fitting, well-formed encapsulation surrounding implant materials, in addition to its ability to be anchored to bone.

Embodiments of the present disclosure include a method for treating bone defects. In accordance with these embodiments, the method includes placing a thermoplastic around the bone defect and molding the thermoplastic.

In some embodiments, a sonotrode coupler and ultrasonic generator are used to mold the thermoplastic. In operation, the ultrasonic generator is connected to a sonotrode system to provide energy to the rod, which vibrates at a rate of 20-70 kHz. This range can be adjusted depending on the thermoplastic used, its thickness, and the distance from the sonotrode tip to the bone.

In some embodiments, the thermoplastic sleeve delivers osteogenic therapeutics. For example, in some embodiments, the thermoplastic sleeve is coated with an osteogenic therapeutic or contains one or more chambers that contain an osteogenic therapeutic.

Embodiments of the present disclosure include a sonotrode coupler for performing the method of treating bone defects. In accordance with these embodiments, the sonotrode coupler includes a rod to transmit localized heat and a plurality of prongs used to ensure the localized heat is delivered uniformly and accurately.

Embodiments of the present disclosure include a system for performing the method of treating bone defects. In accordance with these embodiments, the system includes one or more of each of a thermoplastic sleeve, an ultrasonic generator, and a sonotrode coupler.

In some embodiments, kits are provided that contain one or more or each of a thermoplastic sleeve, an ultrasonic generator, and a sonotrode coupler. In some embodiments, a plurality of thermoplastic sleeves are provided within a kit. The sleeves may differ in dimension such that the kit contains a variety of sizes to accommodate the range of size that may be needed in different settings. In some embodiments, the thermoplastic is provided in sheet form and is modified (e.g., cut or otherwise formed) to a desirable size prior to application to a bone or during application to the bone.

Other aspects of the disclosure will become apparent by consideration of the detailed description and accompanying drawings.

### BRIEF DESCRIPTION OF THE DRAWINGS

FIG. 1 depicts an example of the thermoplastic sleeve joining three pieces of fractured bone.

FIG. 2 depicts a thermoplastic sleeve holding a mineralized collagen matrix on a defect site of a rat femur.

FIG. 3 depicts an example of a perforated thermoplastic sleeve.

FIG. 4 depicts an exemplary system and process to heat shrink thermoplastic tubing.

FIG. 5 depicts examples of perforation patterns for the thermoplastic sleeve.

FIG. 6 depicts an example of the scalability of the method, device, and system.

### INDEX OF ELEMENTS

10 Thermoplastic sleeve
11 Bone piece
12 Bone piece
13 Bone piece
14 Rat femur with defect
15 Thermoplastic sleeve with circular perforations
16 Coupler surface
17 Coupler rod
18 Ultrasonic generator
19 Lower permeability thermoplastic with slit perforation
20 Higher permeability thermoplastic with square perforations
21 Higher permeability thermoplastic with circular perforations
22 Lower permeability thermoplastic with circular perforation
23 Rat skeleton
24 Rabbit skeleton
25 Sheep skeleton
26 Human skeleton

### DETAILED DESCRIPTION

Unless otherwise defined, all technical and scientific terms used herein have the same meaning as commonly understood by one of ordinary skill in the art. In case of conflict, the present document, including definitions, will control. Preferred methods and materials are described below, although methods and materials similar or equivalent to those described herein can be used in practice or testing of the present disclosure. All publications, patent applications, patents and other references mentioned herein are incorporated by reference in their entirety. The materials, methods, and examples disclosed herein are illustrative only and not intended to be limiting.

The terms “comprise(s),” “include(s),” “having,” “has,” “can,” “contain(s),” and variants thereof, as used herein, are intended to be open-ended transitional phrases, terms, or words that do not preclude the possibility of additional acts or structures. The singular forms “a,” “an” and “the” include plural references unless the context clearly dictates otherwise. The present disclosure also contemplates other embodiments “comprising,” “consisting of” and “consisting essentially of,” the embodiments or elements presented herein, whether explicitly set forth or not.

The accompanying drawings, which are incorporated into and constitute a part of this specification, illustrate one or more embodiments of the present application and, together with the detailed description, aid in the explanation of the principles and implementations of the application. The accompanying drawings serve to aid this application, not limit the application.

In some embodiments, the methods described herein allow for the uniform, mechanically stable, and scalable treatment of bone defects. The methods described herein differ from previous mechanical fixation of bone methods in its ability to allow a thermoplastic sleeve to completely enclose a bone defect during treatment. The technology provides the ability to fully contain therapeutics at a site of intended action. This provides for control of geometry of the regenerated bone and prevents unwanted ectopic bone formation in other surrounding tissues outside of the intended site, such as nerves, tendons, and muscles.

The technology finds use with a wide variety of bone defects, including, but not limited to, bone fractures (e.g., oblique fractures, transverse fractures, longitudinal fractures, greenstick fractures, comminuted fractures, segmental fractures, spiral fractures, stress fractures, avulsion fractures, buckle fractures), bone bruises, bone sprains, surgical alteration of bones (e.g., cosmetic, tumor removal, etc.), and bone loss.

In some embodiments, a bone is enclosed using a thermoplastic sleeve an ultrasonic generator, and a sonotrode coupler. In some embodiments, the thermoplastic sleeve is wrapped around the bone defect, the ultrasonic generator provides heat to the sonotrode coupler, and the sonotrode coupler allows for the localized delivery of heat to safely shrink the thermoplastic sleeve in physiological environments.

In some embodiments, the thermoplastic sleeve encloses and stabilizes bone grafting materials at the intended site of bone defects, limiting unwanted fragment motion and unwanted fragment migration out of the defect site.

In some embodiments, the thermoplastic sleeve is perforated thus allowing for communication between bone graft materials and the surrounding physiologic environment thus facilitating vascular ingrowth and exchange of nutrients between the defect site and adjacent tissues. In some embodiments, perforations have slit, square, and/or circular cross-sections, although any suitable shape may be employed. In operation, bone grows through perforations. Higher permeability improves the amount of bone ingrowth. Permeability depends on porosity, orientation, size, distribution, and interconnectivity of the pores. Larger pore size is preferred for cell growth and proliferation as they have greater space for nutrient and oxygen supply. However, the mechanical properties of a material change with increased porosity.

In some embodiments, the thermoplastic sleeve is incorporated with osteogenic therapeutics for controlled, long-term, and/or sustained drug delivery. By way of non-limiting example, osteogenic agents include bone grafts, matrices (e.g., mineralized collagen matrices; woven collagen sponges, e.g., containing calcium triphosphate), cells, growth factors, and/or matrix proteins that promote bone regeneration after implantation. In some embodiments, the incorporation includes coating. In some embodiments, the incorporation includes pocketing the osteogenic therapeutic in internal zones. In some embodiments, the osteogenic therapeutic provides effectiveness upon contact. In some embodiments, the osteogenic therapeutic releases upon thermoforming. In some embodiments, the incorporation allows for a slow release of therapeutics.

In some embodiments, one or more osteogenic agents are added to the bone defect region prior to or during addition of the thermoplastic sleeves such that the agent or agents are contained within the sleeve after thermoforming of the sleeve to the bone region.

In some embodiments, the thermoplastic sleeve is composed of PLA (polylactic acid), PLLA (poly(L-lactide)), PDLLA (poly(DL-lactide)), PGA (polyglycolide or poly(glycolic acid)), polyolefins, polyethylene, and/or polypropylene. In some embodiments, the thermoplastic sleeve comprises a memory polymer (see e.g., Barnes and Verduzco, Soft Matter, 15(5), 870-879, herein incorporated by reference in its entirety).

In some embodiments, the thermoplastic sleeve is composed of polyolefins, polyethylene, and polypropylene and is non-resorbable. In some embodiments, the thermoplastic sleeve is composed of PLA, PLLA, PDLLA, and PGA and is resorbable. In some embodiments, the thermoplastic sleeve is irradiated and stretched. In some embodiments, the thermoplastic sleeve is irradiated with an electron beam resulting in altered mechanical properties of the thermoplastic.

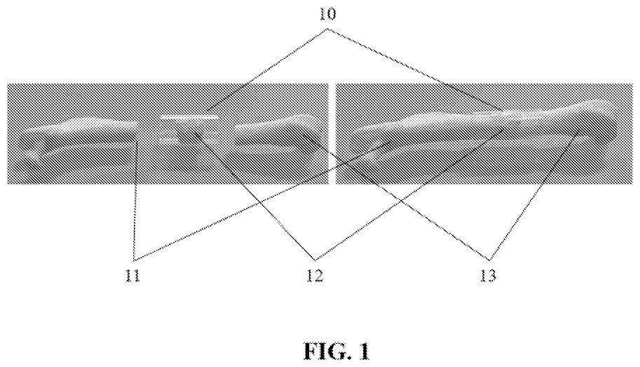

FIG. 1 depicts the thermoplastic sleeve 10 used to connect three bone pieces 11, 12, 13 of a fractured bone. When the thermoplastic 10 is heated (e.g., using an ultrasonic generator and sonotrode couplers), it molds to the bone. Once molded to the bone, the thermoplastic sleeve 10 provides mechanical fixation to the three bone pieces 11, 12, 13 and aids with bone regeneration.

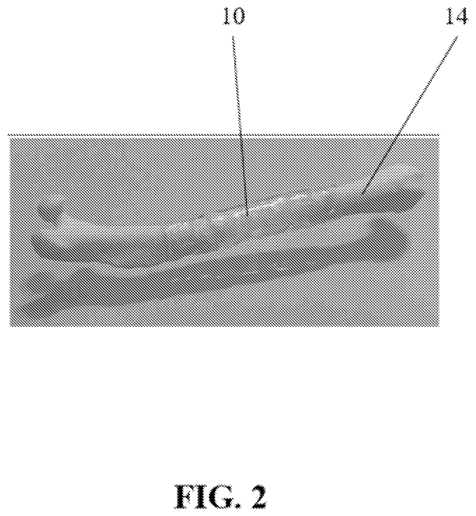

FIG. 2 depicts the thermoplastic sleeve 10 molded to a rat femur 14. The thermoplastic sleeve 10 is molded (e.g., using an ultrasonic generator and sonotrode couplers). The thermoplastic sleeve 10 securely holds a mineralized collagen matrix on the defect site of the bone.

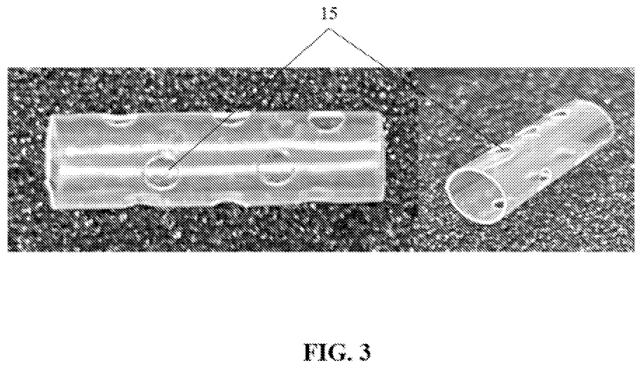

FIG. 3 depicts a thermoplastic sleeve with circular perforations 15. The perforations allow for communication between bone graft materials and the surrounding physiologic environment thus facilitating vascular ingrowth and exchange of nutrients between the defect site and adjacent tissues. This thermoplastic sleeve 15 has lower permeability thus decreasing bone ingrowth but increasing mechanical strength.

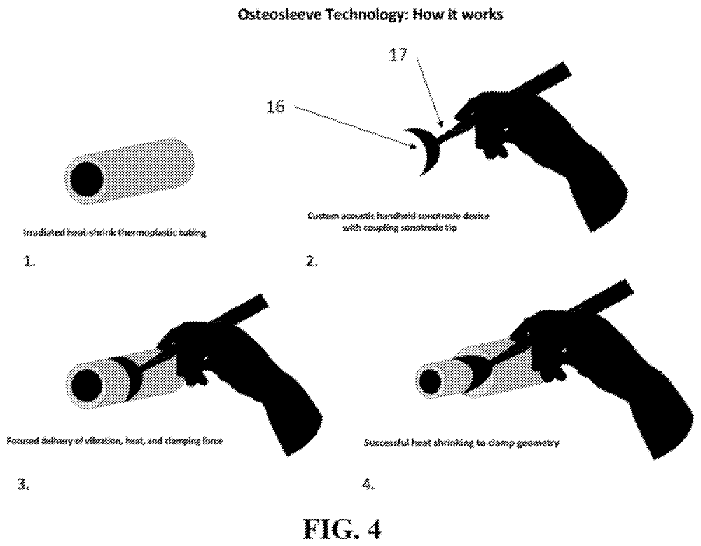

FIG. 4 depicts an exemplary system and process. 1. An irradiated heat-shrink thermoplastic tubing is prepared; 2. A custom acoustic handheld sonotrode device with coupling sonotrope tip is provided. In some embodiments, the sonotrode coupler is composed of a rod 17 and a surface 16 configured to contact the thermoplastic surface (e.g., set of clamps). In some embodiments, the sonotrode coupler works with an ultrasonic generator. The sonotrode coupler converts high-frequency ultrasonic energy into acoustic energy that is used to generate localized heat. In some embodiments, the sonotrode system comprises an ultrasonic welding system comprising a power source, voltage controller, transducer, amplifier, horn and sonotrode tip. 3. The surface 16 of the sonotrode is contacted to the exterior surface of the thermoplastic tubing and provides focused delivery of vibration, heat, and a clamping force. 4. The sonotrode is moved along the surface of the tubing, heat shrinking the thermoplastic tubing based on the clamp geometry.

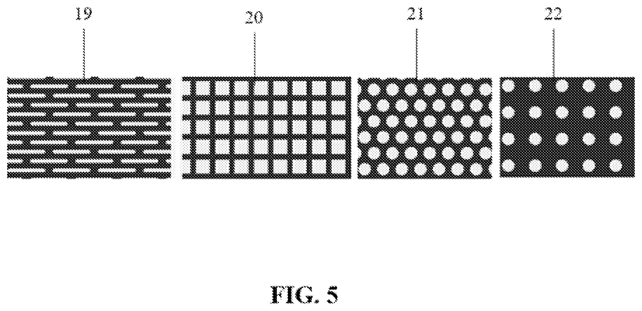

FIG. 5 depicts example perforation patterns that can be applied to the thermoplastic sleeve. The perforations allow for communication between bone graft materials and the surrounding physiologic environment thus facilitating vascular ingrowth and exchange of nutrients between the defect site and adjacent tissues. The thermoplastic with slit perforations 19 has lower permeability due to its small cross-sectional area and low porosity. The thermoplastic with square perforations 20 has higher permeability due to its large cross-sectional area, high distribution, and high porosity. The thermoplastic with circular perforations 21 has higher permeability due to its high porosity and high distribution. The thermoplastic with circular perforations 22 has lower permeability due to its low porosity and low distribution. Lower permeability decreases potential for bone ingrowth but increases mechanical properties. Higher permeability increases potential for bone ingrowth and decreases mechanical properties.

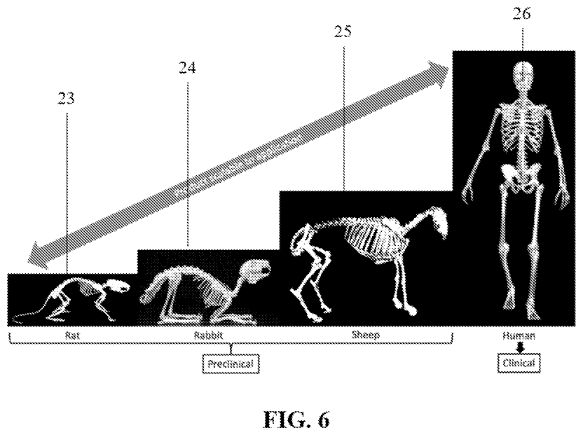

FIG. 6 depicts the scalability of the method. Thermoplastics come in a variety of compositions and sizes. The method can be scaled for small animals and large animals as evidenced by its potential with rats 23, rabbits 24, sheep 25, and humans 26. This device can be used in research (e.g., drug screening), preclinical, and clinical applications.

We/I claim:

1. A method for treating bone defects comprising:
(i) placing a thermoplastic around a bone defect; and
(ii) molding the thermoplastic.

2. The method of claim 1, wherein said molding comprises heating the thermoplastic.

3. The method of claim 2, wherein said heating is provided by one or more of an ultrasonic device, an electrically heated probe, a fluid heated probe, or warm or hot air.

4. The method of claim 1, wherein said molding comprises placing a sonotrode coupler in proximity to said thermoplastic, wherein said sonotrode is coupled to an ultrasonic generator.

5. The method of claim 1, wherein the bone defect is a broken bone.

6. The method of claim 1, wherein the thermoplastic is perforated.

7. The method of claim 1, wherein the thermoplastic is an implantable sleeve.

8. The method of claim 1 wherein the thermoplastic is irradiated.

9. The method of claim 1 wherein the thermoplastic is resorbable.

10. The method of claim 1 wherein the thermoplastic is non-resorbable.

11. The method of claim 1 wherein the thermoplastic comprising one or more osteogenic agent.

12. A sonotrode coupler, comprising:
(i) a rod configured to transmit localized heat; and
(ii) A plurality of prongs shaped to provide mechanical surface contact to a circumference of a thermoplastic wrapped bone surface.

13. A system comprising:
(i) a thermoplastic;
(ii) an ultrasonic generator; and
(iii) a sonotrode coupler of claim 12.
